(19) United States
(12) Patent Application Publication
(10) Pub. No.: US 2020/0206157 A1
(43) Pub. Date: Jul. 2, 2020

(54) COMPOSITION FOR USE IN THE PREVENTION AND TREATMENT OF PATHOLOGIES OF THE CARDIOVASCULAR APPARATUS

(71) Applicant: PHARMANUTRA S.P.A., Pisa (IT)

(72) Inventors: Andrea LACORTE, Pisa (IT); Germano TARANTINO, Pisa (IT); Elisa BRILLI, Pisa (IT)

(21) Appl. No.: 16/631,426

(22) PCT Filed: Jul. 26, 2018
(86) PCT No.: PCT/IB2018/055586

§ 371 Date: Jan. 15, 2020

(30) Foreign Application Priority Data
Jul. 26, 2017 (IT) 102017000085412

(51) Int. Cl. A61K 31/122; A61K 33/06; A61K 33/26; A61K 47/36; A61P 9/00; A23L 33/15

(52) U.S. Cl. CPC A61K 31/122; A61K 33/06; A61K 33/26; A61K 9/0053; A61P 9/00; A23L 33/15; A61K 47/36

(57) ABSTRACT

The present invention relates to a composition comprising or, alternatively, consisting of an effective amount of a mixture comprising at least (a) one lipophilic vitamin, or analogues and derivatives thereof, (b) one magnesium salt and (c) one iron (III) oxide, complex or salt for use in a preventive or curative method for treating cardiovascular disorders and pathologies.

This invention relates to a composition for use in a method of treatment and/or in the prevention of pathologies, diseases or disorders of the cardiovascular apparatus, in particular of pathologies, diseases and disorders linked to vascular calcification.

“Vascular calcification” (VC) is vascular ossification (VC), i.e. the deposition of calcium salts (like, for example calcium phosphate) at the vascular level, which is generally secondary to an alteration of mineralization with consequent transformation of the vessels into tissue similar to bone tissue. VC can affect the tunica intima of the vessel wall (atherosclerosis) and the tunica media (medial calcific sclerosis) involving different proteins both protectively and osteoinductively.

Vascular calcification is an active process of an independent pathology but it is closely associated with a high risk of cardiovascular mortality. From the clinical point of view, vascular calcification leads to hardening of the endothelial wall, causing a decrease in arterial compliance and consequently the development of left ventricular hypertrophy and decreased coronary perfusion. This type of pathology is often found in old people but also in certain types of pathology such as diabetes, aortic stenosis, atherosclerosis and chronic kidney disease.

The latest scientific evidence has shown that vascular calcification is generated following excessive deposition of calcium salts that is mainly due to decreased expression of the Matrix Gla-Protein (MGP). MGP protein is a small protein that is a member of the family of Gla K₂-dependent proteins and its main role is to prevent vascular calcification, owing to its great affinity with calcium ions and to contribute to the organisation of the bone tissue. MGP synthesis was observed and confirmed in different tissues like bone tissue, cartilaginous and cardiac tissue. Further, its expression has been observed also at the levels of the atherosclerotic plaques that may be formed at the vascular level. Of the different proteins involved in metabolizing vascular calcium, MGP plays a dominant role. Although its action has not yet been completely clarified, it has been shown that MGP is a powerful inhibitor of arterial calcification. In fact, subjects affected by Keutel's Syndrome, a hereditary MGP malfunction, are affected by extensive extraosseous calcification. The importance of this protein for vascular health is demonstrated by the fact that until today no effective alternative mechanisms have been disclosed for inhibiting vascular calcification. The MGP protein is a protein that is dependent on vitamin K₂, i.e. a post-translational modification is required for activation, in particular carboxylation, where the co-factor of the carboxylation reaction has been shown to be vitamin K₂. Once activated, the MGP protein can prevent calcium being deposited and crystallized at the level of different organs and tissues, including vascular smooth muscle tissue.

Preventing or retarding vascular calcification is particularly important in patients with chronic kidney disease, in which there is the highest percentual incidence of development of arterial calcification, which is a risk factor for patient survival.

Although vascular calcification is a major risk factor for cardiovascular diseases, no effective treatment has yet been found. That gives rise to the need to be able to use a composition or nutritional complex that can stimulate MGP activity, in particular for long-term administration, to prevent or retard arterial calcification in patients with chronic kidney disease.

One objective of the present invention is to provide a composition for use in a method for treating and/or preventing vascular calcification that is effective, basically free of side effects, and easily tolerable throughout a prolonged treatment for patients with pathologies like diabetes, atherosclerosis and chronic kidney disease.

In response to said need, the present invention provides a composition for use according to the appended claims.

The object of the present invention is a composition (pharmaceutical composition, food supplement or composition for a medical device, briefly composition of the invention) comprising:
an effective amount of a mixture that comprises or, alternatively, consists of (a) a K-group vitamin, (b) an inorganic magnesium salt and (c) an iron (III) oxide, complex or salt; and
pharmaceutical or food grade excipients, additives and/or co-formulants.

An object of the present invention is a composition (composition of the invention) comprising or, alternatively, consisting of:
an effective amount of a mixture that comprises or, alternatively, consists of at least (a) one K-group vitamin, (b) one inorganic magnesium salt and (c) one iron (III) oxide, complex or salt for use in a preventive or curative method for treating vascular calcification and the disorders, diseases and pathologies associated with vascular calcification in a human subject, in which said use comprises administering the composition to said subject, and
pharmaceutical or food grade excipients, additives and/or co-formulants.

In the context of the present invention, the term “composition(s)” is meant to include a pharmaceutical composition, a composition for a food supplement, a composition for a food product or a composition for a medical device.

Preferred embodiments of the present invention will become apparent from the detailed description that follows and are indicated in the appended claims.

Following extensive experimentation, the inventors have devised a nutritional complex consisting of the following substances:
a salt, a complex or a ferrous oxide (Fe(III)), preferably iron pyrophosphate, still more preferably in the form of sucrosomial iron (Sucrosomiale®; Sucrosomial®; registred Trademarks in the name of PHARMANUTRA S.P.A and ALESCO S.R.L.) from Pharmanutra S.p.A.;
a magnesium salt, preferably magnesium chloride or magnesium carbonate and a K-group vitamin like vitamin K₂ (MK-7, vitamin K₂ subtype menaquinone-7).

These elements act synergically, inducing the activation of MGP, and simultaneously, preventing calcium precipitation and crystallization at the level of vascular smooth muscle tissue.

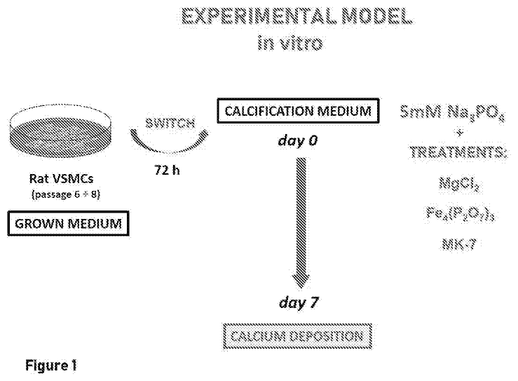

FIG. 1 illustrates the in vitro experimental model for testing the efficacy of the composition according to the present invention in slowing the progression of vascular calcification induced by high levels of inorganic phosphate (Pi).

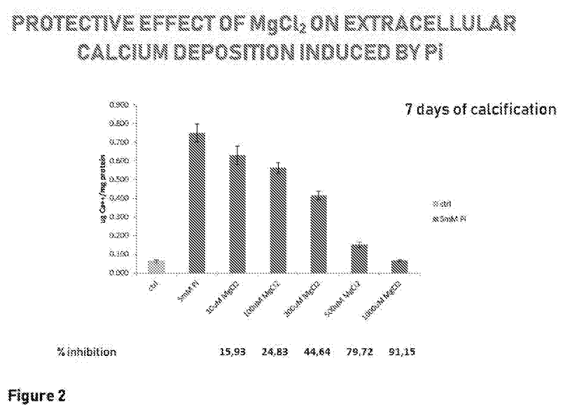

FIG. 2 shows the protective effect of MgCl₂ on calcium deposits in the cell matrix induced by Pi.

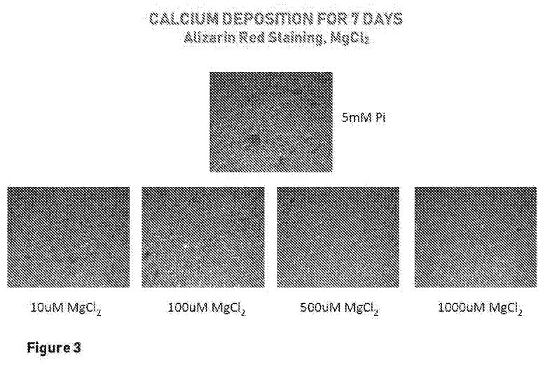

FIG. 3 shows images of cell cultures obtained by a qualitative method (staining of calcium deposits by Alizarin Red Staining) following treatment with MgCl₂, that demonstrate the protective effect of MgCl₂ against calcification induced by Pi in vascular smooth muscle cells (VSMCs).

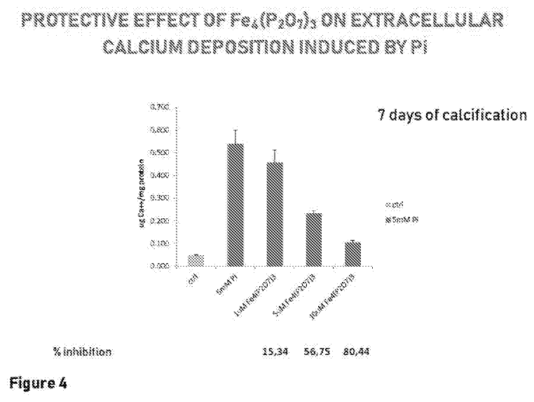

FIG. 4 shows the protective effect of iron pyrophosphate Fe₄(P₂O₇)₃ on extracellular calcium deposits induced by Pi.

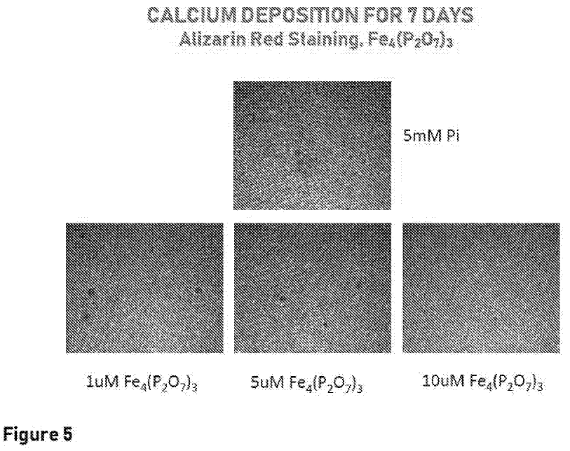

FIG. 5 shows images of a cell culture obtained by a qualitative method (staining of calcium deposits by Alizarin Red Staining) following treatment with iron pyrophosphate.

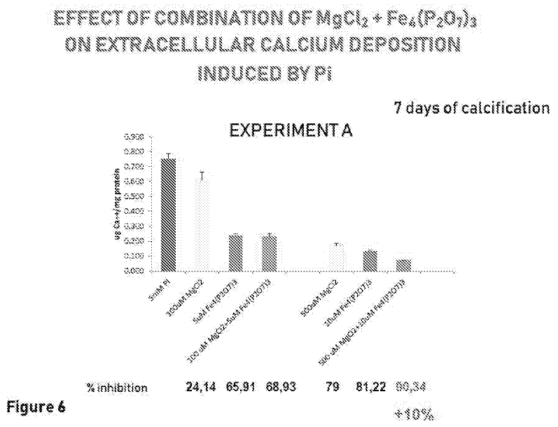

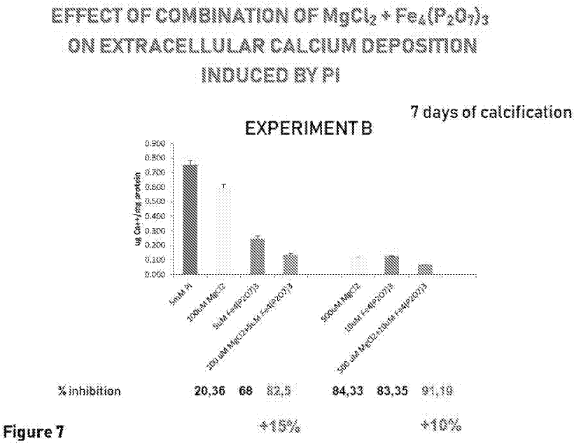

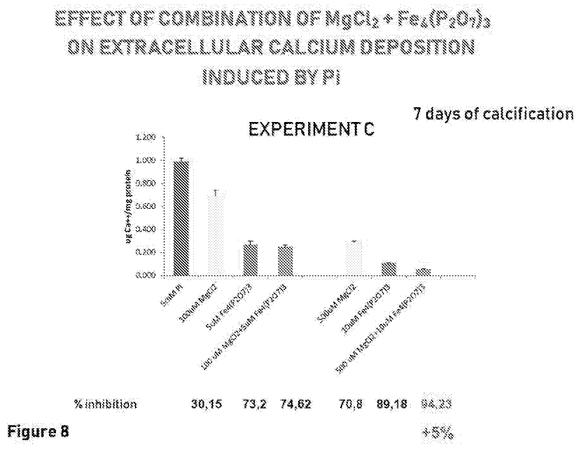

FIGS. 6, 7 and 8 show the effect of the MgCl₂ and iron pyrophosphate combination on extracellular deposits induced by Pi (on the seventh day of calcification).

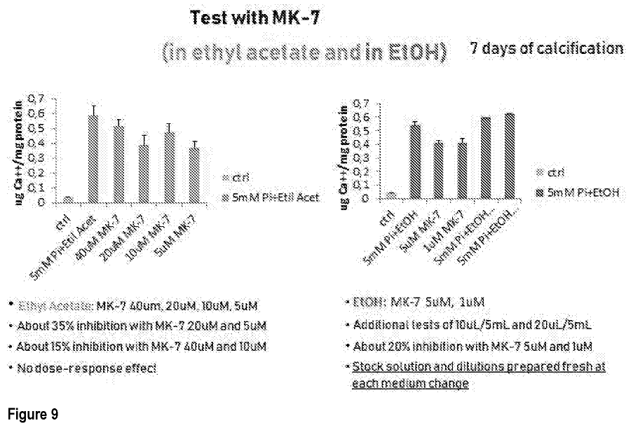

FIG. 9 shows the protective effect of MK-7 against the calcification induced by inorganic phosphate in VSMCs.

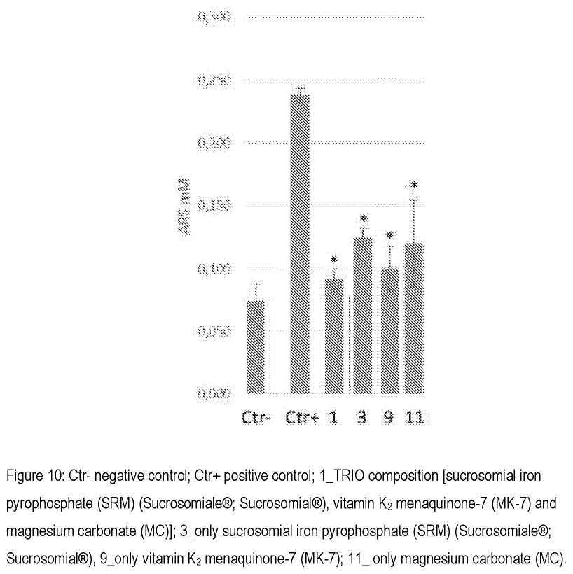

FIG. 10 shows the effect of the combination of sucrosomial iron/MK-7/MgCO₃ (Sucrosomiale®; Sucrosomial®) against the calcification induced by inorganic phosphate in VSMCs cultures with respect to the effect of the single components of the composition.

Within the scope of the present invention, “calcium” means at least one calcium salt produced at the physiological level, like calcium phosphate.

Within the scope of the present invention, “method of treatment” of a pathology or disorder means a therapy aimed at restoring a subject's state of health, maintaining the existing state of health and/or preventing said state of health from worsening.

Within the scope of the present invention, “prevention” of a pathology or disorder means a therapy aimed at preventing the onset of such a pathology or disorder in a subject, also, but not only, as a complication or effect of a pathological condition or pre-existing disorder.

The composition according to the present invention can be for use in human subjects or veterinary use, for example, but without limitation, in pets such as dogs, cats or other mammals. The composition according to the present invention is preferably for use in humans.

Unless specified otherwise, within the scope of the present invention the percentages and amounts of a component in a mixture are intended to refer to the weight of that component relative to the total weight of the mixture.

Within the scope of the present invention, vitamin K means a series of compounds that are 2-methyl-1,4-naphthoquinone derivatives.

The vitamin K family is divided into the following three groups:
Vitamin K₁ or phylloquinone (2-methyl-3-phytyl-1,4-naphthoquinone) of vegetable origin and which is the form that is most common in the diet;
Vitamin K₂, or menaquinones of bacterial origin, is synthesized by the symbiotic bacteria normally found in human intestinal flora, like those belonging to the genus Escherichia (like E. coli); menaquinones differ by the number of isoprenoid units found in the side chain.
Vitamin K₃ or menadione, is liposoluble, is of synthetic origin and its hydrosoluble bisulphite derivative.

In one preferred embodiment, the composition according to the present invention comprises a K-group vitamin of the vitamin K₂ group, in its 9 subtypes, its derivatives or analogues.

Vitamin K₂, the main storage form in animals, has several subtypes, which differ in isoprenoid chain length. These vitamin K₂ homologues are called menaquinones (MK), and are characterized by the number of isoprenoid residues in their side chains. Menaquinones are abbreviated MK-n, where M stands for menaquinones, K stands for vitamin K and the n represents the number of isoprenoid side chain residues. For example, menaquinone-4 (abbreviated MK-4) has four isoprene residues in its side chain. Menachinone-4 (also known as menatetrenone from its four isoprene residues) is the most common type of vitamin K₂ in animal products since MK-4 is normally synthesized from vitamin K₁ in certain animal tissues (arterial walls, pancreas, and testes) by replacement of the phytyl tail with an unsaturated geranylgeranyl tail containing four isoprene units, thus yielding menaquinone-4. This homologue of vitamin K₂ may have enzyme functions distinct from those of vitamin K₁.

Menaquinone-7 (MK-7) is different from MK-4 in that it is not produced by human tissue. MK-7 may be converted from phylloquinone (K₁) in the colon by Escherichia coli bacteria. However, menaquinones synthesized by bacteria (MK-7) appear to contribute minimally to overall vitamin K status. MK-4 and MK-7 are both approved in the United States for use in dietary supplements for bone health.

In one particularly preferred embodiment, the composition according to the present invention comprises vitamin K₂, an MK7 homologue.

The composition according to the present invention comprises, in addition to a K-group vitamin like vitamin K₂, at least one inorganic magnesium salt that is suitable for being taken by human subjects or for veterinary use.

By way of non-limiting example, said salt can be magnesium oxide, magnesium carbonate, magnesium chloride or mixtures thereof.

Preferably, in the composition according to the present invention, the inorganic magnesium salt is one of magnesium oxide, magnesium carbonate and magnesium chloride, preferably magnesium oxide or magnesium carbonate.

In an alternative embodiment, the composition according to the invention contains a salt or organic magnesium complex like magnesium bisglycinate, magnesium citrate, magnesium pidolate, or mixtures thereof. Said organic magnesium salt can be present in the composition of the invention in replacement of the inorganic magnesium salt or, alternatively, in addition to the inorganic magnesium salt.

In an alternative embodiment, the composition according to the invention comprises sucrosomial magnesium (Sucrosomiale®; Sucrosomial®), which consists of a magnesium oxide or an inorganic magnesium salt, lecithin, sucrester, optionally rice starch, optionally pregelatinized, and tricalcium phosphate. Said sucrosomial magnesium (Sucrosomiale®; Sucrosomial®) can be present in the composition of the invention in place of the inorganic magnesium salt or of the organic magnesium salt or, alternatively, in addition to the inorganic magnesium salt or to the organic magnesium salt.

The composition according to the present invention comprises, in addition to a K-group vitamin and to an inorganic magnesium salt, at least one iron (Fe(III)) oxide, complex or salt, that is suitable for being taken by human subjects or for veterinary use.

By way of non-limiting example, said salt can be organic or inorganic, like chloride, sulphate, pyrophosphate, citrate, bisglycinate, fumarate, gluconate, ascorbate, polymaltose and mixtures thereof. Within the scope of the present invention, “complex” means a coordination complex formed by one or more iron atoms and one or more atoms, ions or molecules that surround the complex, at least partially, without forming an ionic or co-valent bond (binders or ligands).

Preferably, the composition according to the invention comprises at least one iron (III) salt, more preferably said salt is iron (III) pyrophosphate (Fe₄(P₂O₇)₃).

In one preferred embodiment, in the composition according to the invention the (iron (III)) salt, oxide or complex is in “sucrosomial” (Sucrosomiale®; Sucrosomial®) form, i.e. in the form of a complex formed by sucresters and lecithin, that facilitate conveying and absorption by the subject.

The term “sucrosomial iron” (Sucrosomiale®; Sucrosomial®) in the context of the present invention indicates a formulation of an aforesaid iron mineral, such as iron (III) salt, oxide or complex, in which said iron mineral is processed and formulated by the preparation methods disclosed below. Preferably, when an iron (III) salt, like for example pyrophosphate iron (III), is processed and formulated together with other substances, by the preparation methods disclosed above, a sucrosomial iron (Sucrosomiale®; Sucrosomial®) is obtained.

In other words, in the composition according to the invention the iron (III) salt, oxide or complex is formulated in association with sucrose fatty acid esters or sucresters (E473), lecithin and, optionally, gelatinized or pregelatinized starch.

In the context of the present invention, the terms “iron (III) formulated in association with sucresters, lecithin” and “iron (III) in association with sucresters, lecithin” have the same meaning.

By way of non-limiting example, when the iron salt, oxide or complex is in “sucrosomial” (Sucrosomiale®; Sucrosomial®) form in the composition of the present invention it can be prepared according to one of the methods disclosed in WO 2014/009806 A1, which is included here for reference. Specifically, a first preparation method for preparing “sucrosomial iron” in solid form is disclosed in WO 2014/009806 A1 from page 7 line 1 to page 8, line 20, included in this application for reference purposes, a second preparation method for preparing “sucrosomial iron” in solid form is disclosed from page 8 line 22 to page 10 line 21, included in this application for reference purposes. Lastly, a method for preparing “sucrosomial iron” in liquid form is disclosed in WO 2014/009806 A1 from page 12, line 21 to page 14, line 3, included in this application for reference purposes.

In one preferred embodiment, the composition according to the invention comprises:
a mixture that comprises or, alternatively, consists of:

(a) a K-group vitamin, preferably vitamin K₂

(b) an inorganic magnesium salt, preferably magnesium carbonate, and

(c) an iron (III) salt, oxide or complex in sucrosomial (Sucrosomiale®; Sucrosomial®) form (as defined in the present invention), i.e. an iron (III) salt, oxide or complex formulated in association with sucrose fatty acid esters or sucresters, lecithin and, optionally, gelatinized or pregelatinized starch of vegetable origin; and
pharmaceutical or food grade excipients, additives and/or co-formulants.

The aforesaid composition is for use in a preventive or curative method for treating vascular calcification and disorders, diseases and pathologies associated therewith.

It has been found that the iron (iron III)) salt or oxide or complex in sucrosomial (Sucrosomiale®; Sucrosomial®) form is more palatable and is easily absorbed also by particularly sensitive subjects, like pregnant women.

Said sucrosomial iron (III) (Sucrosomiale®; Sucrosomial®), comprises, in association with said iron (III) salt, oxide or complex, a lecithin as disclosed below.

Lecithin is a food additive—E322 (directive 95/2/CE of 20.2.95 published in issue L61 of 18.3.95 of the Official Journal). Lecithin, owing to its chemical/physical properties, primarily performs an emulsifying function and, also being rich in natural antioxidant substances, also performs a secondary antioxidant function. Directive 2008/84/CE of 27 Aug. 2008, (published in issue L253 of the Official Journal of the European Community), sets the purity requirements that lecithin must have to be considered fit for human consumption (E322): Insoluble in acetone (practically the active part of the lecithin): 60% min.; humidity 2% max.; acidity number 35 max.; peroxide number: 10 max.; insoluble in toluene (practically); impurity): 0.3% max.

From the chemical point of view, lecithin is a mixture di phosphoric acid, choline, fatty acids, glycerol, glycolips, triglycerides and phospholipids. Phospholipids are the main components; they derive from the structure of the triglycerides, where a fatty acid is replaced by a phosphate group that gives a negative load to the molecule and thus polarity; this molecule has the generic name of phosphatide. A more complex organic molecule, generally serine, choline, ethanolamine, inositol or a single hydrogen atom is linked to the phosphate group by an ester bond, giving rise to a phospholipid known, respectively, as phosphatidylserine, phosphatidylcholine, phosphatidylethanolamine, phosphatidylinositol or phosphatidic acid. Phosphatidylcholine is often indicated as a lecithin in the strictest sense of the term. Phospholipids are characterized by a hydrosoluble polar head, which dissolves well in water, whereas the two saturate fatty acids constitute the two apolar tails that are not hydrosoluble but are lipophile. Molecules of this type are called amphipathic molecules and in the presence of water and fat they are distributed between the fat and the water molecule, emulsifying them. Lecithin is a natural emulsifier.

The composition of the present invention does not contain a hydrolized lecithin or a lecithin hydrolized by enzymes.

The lecithin used is a non-hydrolyzed powder lecithin and can be chosen from a sunflower or maize or soya lecithin. The lecithin used is a lecithin in powder form having a water content comprised between 1.5 and 4.5%, preferably between 2 and 4%, still more preferably between 2.5 and 3.5%. Advantageously, the lecithin used is a sunflower powder lecithin.

In one embodiment, the sunflower lecithin contains an amount by weight of glucose comprised between 20 and 60%, preferably between 30 and 50%, for example about 45% as in, without restriction, the product Lecico Sun™ CG 450 of the company Lecico GmbH—Germany or Lecifis Sun™ CG 45M007501 of Alesco S.r.l.

A sunflower lecithin usable in the context of the present invention can have the following composition by weight (chemical-physical analysis): sunflower lecithin from 40 to 50%, carbohydrates from 40 to 50% (for example approximately 42%), proteins from 6 to 10%, ash content from 3 to 8%, humidity from 2 to 5% and another flowing agent from 0.5 to 1.5%.

The lecithin is present in the solid composition of the present invention in an amount by weight comprised between 0.1 and 1.5%, preferably between 0.4 and 1.0%, still more preferably between 0.50 and 0.8%. Advantageously, the iron (III) salt is iron pyrophosphate and the lecithin is sunflower and/or maize lecithin.

Said sucrosomial iron (III) (Sucrosomiale®; Sucrosomial®) comprises in association with said iron (III) salt, oxide or complex, in addition to said lecithin disclosed above, a sucrester as disclosed below.

The solid or liquid composition of the present invention further comprises, in association with a lecithin disclosed above, a sucrose ester or sucrester.

The sucresters are obtained by the esterification of the fatty acids or by transesterification of the methyl esters of the fatty acids with carbohydrates, in general sucrose and other polysaccharides, for which reason they are also known as fatty acid sucrose esters (alternatively known as sucrose fatty acid esters or carbohydrates fatty acid esters; briefly sucrose esters). The physicochemical properties of these compounds depend on the number and type of esterified fatty acids. The code E473 indicates that the sucresters are food additives permitted by European legislation and subject to Italian Ministerial Decree D.M. 209 of 27 Feb. 1996. They are essentially emulsifiers and are added in order to determine greater stabilization of a water phase with a fatty acid phase.

The sucrose esters are sucresters (E473) and are used in the composition of the present invention with non-ionic surfactants having an HLB value of about 14-18, advantageously with an HLB value of about 15 or 16, and are used as emulsifiers.

In one embodiment, the sucrester E473 contains 70% monoesters, obtained by esterification of the sucrose with fatty acids of vegetable origin (stearic and palmitic acid).

A sucrester usable in the context of the present invention can have the following composition by weight: total esters content of at least 90%; free fatty acids content (like oleic acid) not above 3%; free sucrose content not above 2%; humidity not above 4%; acidity value not exceeding 5.

The sucrose esters (sucrose fatty acid esters) or sucresters are present in the solid composition in an amount by weight comprised between 10 to 20%, preferably between 12.5 and 18.5%, still more preferably between 16 and 18.0%.

The solid or liquid composition for oral use of the present invention does not contain a diglycerol fatty acid ester.

The solid or liquid composition of the present invention, in addition to a K-group vitamin and to an inorganic magnesium salt, preferably comprises iron (III) salts, complexes or oxides, a lecithin E322 (not hydrolized and not hydrolized by enzymes) and sucrose esters or sucresters E473 in an amount by weight as indicated above (i.e. a sucrosomial iron (Sucrosomiale®; Sucrosomial®)).

In one preferred embodiment, the composition of the present invention comprises an effective amount of a mixture that comprises or, alternatively, consists of

(a) a K-group vitamin, preferably vitamin K₂

(b) an inorganic magnesium salt, preferably magnesium carbonate, and

(c) iron (III) pyrophosphate formulated in association with a lecithin E322 (not hydrolized and not hydrolized enzymatically), preferably sunflower, and sucrose fatty acid esters E473 or sucresters E473 (sucrosomial iron (Sucrosomiale®; Sucrosomial®) of iron pyrophosphate), in an amount by weight as indicated above.

Said sucrosomialiron (III) (Sucrosomiale®; Sucrosomial®) can comprise, in association with an iron (III) salt, complex or oxide, in addition to said sucresters and said lecithin, also a gelatinized or pregelatinized starch of vegetable origin as disclosed below.

In one preferred embodiment, the solid or liquid composition of the present invention can further comprise a gelatinized or pregelatinized starch of vegetable origin.

The starch of vegetable origin is chosen from rice or maize starch. Advantageously, the starch is a rice starch. Advantageously, the rice starch is a native gelatinized or pregelatinized rice starch.

A pregelatinized rice starch that is usable in the composition of the present invention can have the following chemical/physical features: humidity not above 7%; proteins content not exceeding 1%; ash content not exceeding 1%; pH (10% solution) comprised between 5.5 and 7.5, density 0.40-0.48 g/cm³; 97% minimum starch content and fatty acids content not exceeding 0.1%. The gelatinized or pregelatinized starch of vegetable origin, is present in the solid composition in an amount by weight comprised between 15 and 40%, preferably between 20 and 35, still more preferably between 25 and 30% of the total weight of the sucrosomial iron (Sucrosomiale®; Sucrosomial®) (i.e. iron complex oxide or salt+lecithin+sucresters E 473) present.

By way of non-limiting example, said composition comprising starch of vegetable origin can be prepared according to one of the methods disclosed in WO 2014/009806 A1, which is included here for reference (see paragraphs cited previously).

A first method, by way of non-limiting example, enables a solid composition to be prepared comprising or, alternatively, consisting of an iron (III) salt, a lecithin and a gelatinized or pregelatinized starch of vegetable origin.

Said first method of the present invention comprises or, alternatively, consists of a series of processing steps by which the iron salt is covered, or wrapped, or encapsulated, with said lecithin and/or said vegetable starch.

The iron (III) pyrophosphate salt is placed in contact with said lecithin and/or said vegetable starch. The salt in solid powder or granule state has a water content of less than 3% by weight.

The iron salt containing the iron (III) cation is used in an amount by weight comprised between 50 and 90%, preferably between 60 and 80%, still more preferably between 70 and 75%.

Advantageously, the salt is an iron (III) salt. Advantageously, the iron salt (III) is iron pyrophosphate having the above features.

The lecithin used has the above features.

The processing time for the various components is comprised between 1 and 60 minutes, preferably between 10 and 50 minutes, still more preferably between 20 and 40 minutes.

The lecithin used can be chosen from a sunflower or maize or soya lecithin. The lecithin used is a lecithin in powder form having a water content comprised between 1.5 and 4.5%, preferably between 2 and 4%, still more preferably between 2.5 and 3.5%. Advantageously, the lecithin used is a sunflower powder lecithin.

In said first method for preparing the solid composition for oral use of the present invention, a hydrolized lecithin is not used and neither is a lecithin hydrolized in an enzymatic manner used.

The lecithin is present in the solid composition of the present invention in an amount by weight comprised between 0.1 and 1.5%, preferably between 0.4 and 1.0%, still more preferably between 0.50 and 0.8%.

The lecithin, brought into contact with said iron salt, is distributed evenly over said salt.

The gelatinized or pregelatinized starch of vegetable origin is chosen from a rice starch or a maize starch. Advantageously, the starch is a rice starch. Advantageously, the rice starch is a gelatinized or pregelatinized native rice starch. The starch of vegetable origin has the features disclosed above.

The starch is present in the solid composition of the present invention in an amount by weight comprised between 15 and 40%, preferably between 20 and 35%, still more preferably between 25 and 30%.

The starch in gelatinized or pregelatinized starch form has the advantage of being more fluid and flowable and is able to be dosed accurately without giving rise to errors or weight variations. Further, it is distributed more uniformly and homogeneously. Lastly, the pregelatinized starch improves the bioavailability of the salt and accordingly the bioavailability of the cation contained in said salt because the obtained compound dissolves better at temperatures comprised between 15 and 30° C. (pressure 1 atmosphere), preferably between 20 and 25° C., still more preferably between 18 and 23° C.

At the end of said first preparation method, a solid composition of the present invention is obtained that comprises or, alternatively, consists of iron (III) salts, a lecithin (not hydrolized and not hydrolized enzymatically) and a pregelatinized starch of vegetable origin, in the amounts by weight indicated above. In particular, from said first preparation method a solid composition of the present invention is obtained that comprises or, alternatively, consists of pyrophosphate iron (III) salts, a sunflower lecithin (not hydrolized and not hydrolized enzymatically) and a pregelatinized rice starch, in the amounts by weight indicated above.

The Applicant has found that, to improve further the bioavailability of the salt and, thus, the bioavailability of the cation contained in said salt, the amount by weight of lecithin to be used in the process for preparing the solid composition of the present invention can be reduced in quantity as much as possible.

Further, the Applicant has found that to improve further the bioavailability of the salt and, thus, the bioavailability of the cation contained in said salt, in association with a reduced amount by weight of lecithin, it is preferable to use an amount by weight of sucrose esters or sucresters.

The composition of the present invention can be prepared, without restrictions, with a second method for preparing a composition in solid form.

A second method of the present invention relates to preparing a solid composition comprising or, alternatively, consisting of an iron salt, sucrose esters or sucresters, a lecithin and a gelatinized or pregelatinized starch.

Said second method of the present invention comprises or, alternatively, consists of a technology designed to create around the iron a coating or encapsulation so as to improve the stability and bioavailability of the cation.

In practice, said second method provides the formation of agglomerates or granules comprising the iron salt, the sucrose esters or sucresters, the lecithin and a gelatinized or pregelatinized starch. All these components have the features specified above.

Sucrose esters or sucresters and lecithin have the function of increasing consumption of salt and thus of the iron cation contained in said salt. Mixing lecithin and starch gives rise to the formation of “chimeric” agglomerates that are able to protect and screen the iron cation contained in said salt from the gastric acid.

The iron salt containing the iron (III) cation is used in an amount by weight comprised between 30 and 70%, preferably between 40 and 60%, still more preferably between 50 and 55%.

The iron salt used has the above features. Advantageously, the salt is an iron (III) salt. Advantageously, the iron (III) salt is iron pyrophosphate.

The processing time is comprised between 1 and 60 minutes, preferably between 10 and 50 minutes, still more preferably between 20 and 40 minutes.

The sucrose esters or sucresters are present in an amount by weight comprised between 10 and 30%, preferably between 15 and 25%, still more preferably between 16 and 20%.

The lecithin used is a maize or sunflower or soya lecithin. The lecithin used is a lecithin in powder form having a water content comprised between 1.5 and 4.5%, preferably between 2 and 4%, still more preferably between 2.5 and 3.5%. Advantageously, the lecithin used is a sunflower powder lecithin. The lecithin used has the above features.

The lecithin is present in an amount by weight comprised between 0.1 and 1.5%, preferably between 0.4 and 1.0%, still more preferably between 0.5 and 0.8%.

When the lecithin is brought into contact with said granule or powder, the lecithin is distributed over the outer surface of the granules or powder.

Subsequently, a gelatinized or pregelatinized starch of vegetable origin is used that is chosen from a rice starch or a maize starch. Advantageously, the starch is a rice starch. Advantageously, the rice starch is a native gelatinized or pregelatinized rice starch. The starch used has the above features.

The starch is present in the solid composition of the present invention in an amount by weight comprised between 15 and 40%, preferably between 20 and 35%, still more preferably between 25 and 30%.

The gelatinized or pregelatinized starch is prepared with apparatuses and techniques known to the person skilled in the art.

In one embodiment, the lecithin is used in an amount by weight comprised between 0.48 and 0.62%, whereas the sucrose esters or sucresters are used in an amount by weight comprised between 16.5 and 18.5% of the weight of the final solid composition of the present invention. These combinations enable the bioavailability of cation contained in said salt in question to be increased.

At the end of said second preparation method, a solid composition of the present invention is obtained that comprises or, alternatively, consists of iron (III) salts, sucrose esters or sucresters, a lecithin (not hydrolized and not hydrolized enzymatically) and a pregelatinized starch of vegetable origin, in the amounts by weight indicated above.

In particular, by said second preparation method, a solid composition of the present invention is obtained that comprises or, alternatively, consists of iron (III) pyrophosphate salts, sucrose esters or sucresters E473, a sunflower lecithin (not hydrolized and not hydrolized enzymatically) and a pregelatinized rice starch, in the amounts by weight indicated above.

The solid composition of the present invention has a granulometry (defined as average granulometry measured with available apparatuses and techniques) that is comprised between 8 and 16 microns, preferably between 10 and 14 microns, still more preferably between 11 and 13 microns. The solid composition of the present invention has an iron (III) content comprised between 60 mg/g and 140 mg/g, preferably between 80 mg/g and 120 mg/g, still more preferably between 90 and 110 mg/g.

The liquid composition of the present invention comprises or, alternatively, consists of water, iron salts, a lecithin, sucrose esters or sucresters and a guar gum. All these components have the features specified above.

The liquid composition of the present invention does not contain a hydrolized lecithin or a lecithin hydrolized by enzymes.

The liquid composition of the present invention does not contain a diglycerol fatty acid ester.

The liquid composition of the present invention has a viscosity (measured in standard conditions and by known apparatuses and techniques) comprised between 1.01 and 1.12 g/ml, preferably between 1.02 and 1.10 g/ml, still more preferably between 1.03 and 1.08 g/ml.

The liquid composition of the present invention comprises iron salts, having the characteristics set out above. The iron salts are iron III salts (iron salt (III)). Advantageously, the iron (III) salt is iron pyrophosphate.

The liquid composition of the present invention contains an iron salt in an amount by weight comprised between 1 and 10%, preferably between 2 and 8%, still more preferably between 4 and 6%, of the weight of the liquid composition.

The liquid composition of the present invention further comprises sucrose esters or sucresters, having the features disclosed above.

The sucrose esters or sucresters E473 are present in said liquid composition of the present invention in an amount by weight comprised between 0.10 and 5%, preferably between 0.5 and 4%, still more preferably between 1 and 3%, of the weight of the liquid composition.

The liquid composition of the present invention further comprises a lecithin, having the features specified above.

The E322 lecithin used can be chosen from a sunflower or maize or soya lecithin. Advantageously, the lecithin used is a sunflower lecithin.

In one embodiment, the sunflower lecithin contains an amount by weight of glucose comprised between 20 and 60%, preferably between 30 and 50%, for example 45% as in the product Lecico Sun™ CG 450 of the company Lecico GmbH—Germany or Lecifis Sun™ CG 45M007501 of Alesco S.r.l.

A sunflower lecithin usable in the context of the present invention can have the following composition by weight (chemical-physical analysis): sunflower lecithin from 40 to 50%, carbohydrates from 40 to 50% (for example, 42% carbohydrates), proteins from 6 to 10%, ash from 3 to 8%, humidity from 2 to 5% and another flowing agent from 0.5 to 1.5%.

The lecithin is present in said liquid composition in an amount by weight comprised between 0.1 and 4%, preferably between 0.5 to 3.5%, still more preferably between 1.5 and 2.5%, of the weight of the liquid composition.

The liquid composition of the present invention further comprises a guar gum.

The guar gum is present in said liquid composition of the present invention in an amount by weight comprised between 0.1 and 5%, preferably between 0.2 and 4%, still more preferably between 0.4 and 2% of the weight of the liquid composition.

In one embodiment, the guar gum is chosen from those commercially available and has a viscosity (cPs, 2 hours) comprised between 3000-4500, preferably between 3500 and 4000; starch free; with a content of substances that are insoluble in acid comprised between 5 and 9, preferably between 6 and 8, for example 7; with R.U.A. comprised between 2.5 and 4%, preferably between 3 and 3.5% and granulometry comprised between 100 and 300, preferably between 150 and 250, for example 200.

In one embodiment, the liquid iron composition according to the present invention is prepared from water. The water is present in an amount by weight of 90%, or 92%, or 94%. The water continues to be stirred at a temperature comprised between 15 and 45° C. (pressure 1 atmosphere), preferably between 20 and 35° C., still more preferably between 25 and 30° C.

Subsequently, the sucrose esters or sucresters, the lecithin, the guar gum and the iron (III) salts (having features disclosed above) are added to the amounts specified below. The sucrose esters or sucresters are present in an amount by weight comprised between 0.10 and 5%, preferably between 0.5 and 4%, still more preferably between 1 and 3%, of the weight of the liquid composition.

The water and sucrose esters or sucresters give rise to a clear solution/suspension at a temperature comprised between 15 and 45° C. (pressure 1 atmosphere), preferably between 20 and 35° C., still more preferably between 25 and 30° C.

The processing time is comprised between 1 to 60 minutes, preferably between 10 and 50 minutes, still more preferably between 20 and 40 minutes.

The lecithin (having the features disclosed above) is used in an amount by weight comprised between 0.1 and 4%, preferably between 0.5 and 3.5%, still more preferably between 1.5 to 2.5%, of the weight of the liquid composition.

The water, the sucrose esters or sucresters and the lecithin give rise to a clear solution/suspension at a temperature comprised between 15 and 45° C. (pressure 1 atmosphere), preferably between 20 and 35° C., still more preferably between 25 and 30° C.

The guar gum (having the features disclosed above) is used in an amount by weight comprised between 0.1 and 5%, preferably between 0.2 and 4%, still more preferably between 0.4 and 2%, of the weight of the liquid composition.

The water, the sucrose esters or sucresters, the lecithin and the guar gum give rise to a clear solution/suspension at a temperature comprised between 15 and 45° C. (pressure 1 atmosphere), preferably between 20 and 35° C., still more preferably between 25 and 30° C.

Said iron salt (having the features disclosed above) in an amount by weight comprised between 1 and 10%, preferably between 2 and 8%, still more preferably between 4 and 6%, of the weight of the liquid composition.

At the end of processing, an opalescent solution or homogeneous suspension is obtained. The operating temperature is comprised between 15 and 45° C. (pressure 1 atmosphere), preferably between 20 and 35° C., still more preferably between 25 and 30° C. The processing time is comprised between 1 and 60 minutes, preferably between 20 and 50 minutes, still more preferably between 30 and 40 minutes.

Subsequently, the liquid composition is subjected to heat treatment, for example pasteurisation. In practice, the liquid composition that is at a temperature comprised between 20 and 25° C. is heated to a temperature of approximately 110° C. and is then cooled to a temperature of about 25-30° C. The heat treatment phase lasts between 1 and 3 minutes.

At the end of said process for preparing said liquid composition the liquid composition of the present invention is obtained that comprises or, alternatively, consists of water, iron (III) salts, sucrose esters or sucresters, a lecithin (not hydrolized and not hydrolized enzymatically) and a guar gum, at the amounts by weight indicated above.

In particular, from said preparation process the liquid composition is obtained comprising sucrosomial iron (Sucrosomiale®; Sucrosomial®) that comprises or, alternatively, consists of water, pyrophosphate iron (III) salts, sucrose esters or sucresters E473, a sunflower lecithin (not hydrolized and not hydrolized enzymatically) and a guar gum, in the amounts by weight indicated above.

The solid compositions for oral use of the present invention, obtained by said first and second method, are raw materials in solid state (granules or agglomerates or powders) which are subsequently mixed with pharmaceutically acceptable additives and excipients to give rise to pharmaceutical forms for oral use such as pills, tablets, capsules, sachets.

The liquid composition for oral use of the present invention is mixed with pharmaceutically acceptable aromas, excipients and additives to give rise to a liquid syrup or suspension for oral use.

In one embodiment, the solid or liquid composition of the present invention comprises, in addition to a K-group vitamin and to an inorganic magnesium salt, at least one iron (III) salt or oxide or complex, a lecithin E322 (not hydrolized and not hydrolized enzymatically), sucrose esters or sucresters E473 and a starch of vegetable origin (sucrosomial iron (Sucrosomiale®; Sucrosomial®)), in the amounts by weight indicated above.

In one embodiment, the solid or liquid composition of the present invention comprises, in addition to a K-group vitamin and to an inorganic magnesium salt, at least one iron (III) pyrophosphate salt or oxide, a sunflower lecithin (not hydrolized and not hydrolized enzymatically), sucrose esters or sucresters E473 and a pregelatinized rice starch (sucrosomial iron (Sucrosomiale®; Sucrosomial®)), in the amounts by weight indicated above.

In one preferred embodiment, the composition of the present invention comprises an effective amount of a mixture that comprises or, alternatively, consists of

(a) a K-group vitamin, preferably vitamin K₂

(b) an inorganic magnesium salt, preferably magnesium carbonate, and

(c) an iron (III) salt, oxide or complex, preferably iron (III) pyrophosphate formulated in association with a lecithin E322 (not hydrolized and not hydrolized enzymatically), preferably of sunflower, sucrose fatty acid esters E473 or sucresters E473 and a gelatinized or pregelatinized starch of vegetable origin (sucrosomial iron(Sucrosomiale®; Sucrosomial®)), in an amount by weight as indicated above.

In one preferred embodiment, the composition of the present invention comprises an effective amount of a mixture that comprises or, alternatively, consists of

(a) vitamin K₂, preferably subtype menaquinone-7 (MK7)

(b) magnesium carbonate, and

(c) iron (III) pyrophosphate formulated in association with sunflower lecithin E322, sucrose fatty acid esters E473 or sucresters E473 and a pregelatinized starch of vegetable origin (sucrosomial iron(Sucrosomiale®; Sucrosomial®)).

In one preferred embodiment, the composition of the present invention comprises the three components (a), (b), (c) of the mixture in the following amounts by weight:

(a) vitamin K₂ subtype menaquinone-7 (MK7) in amount by weight comprised in a range between 30 mcg and 200 micrograms (mcg) with respect to the total weight of the composition, preferably between 50 mcg and 150 mcg, more preferably between 80 mcg and 120 mcg;

(b) magnesium carbonate in an amount by weight comprised in a range between 30 mg and 200 mg with respect to the total weight of the composition, preferably between 50 mg to 150 mg, more preferably between 80 mg to 120 mg; and (c) iron (III) pyrophosphate formulated in association with sunflower lecithin E322, sucrose fatty acid esters E473 or sucresters E473 and a pregelatinized starch of vegetable origin (sucrosomial iron (Sucrosomiale®; Sucrosomial®)) in an amount by weight of sucrosomial iron (Sucrosomiale®; Sucrosomial®) comprised in a range between 1 mg and 60 mg with respect to the total weight of the composition, preferably between 10 mg and 50 mg, more preferably between 20 mg and 40 mg.

Preferably, the aforesaid quantities of the three components (a), (b) and (c) are to be understood as the amount by weight of the three components to be administered to a subject as a daily dose.

It is understood that the treatment according to the invention comprises said administration of the active ingredients (a)-(c), and, if present, of the other ingredients, can take place simultaneously, for example in a single formulation, or in a rapid sequence, for example through two or more formulations taken by the subject in any order, in a close sequence over time (e.g. within 1 to 10 minutes) in two distinct compositions.

In one preferred embodiment, the composition (composition of the invention) is administered to the subject orally, for example in the form of a pill or tablet, which may also be coated, capsule, solution, suspension, syrup, foodstuff containing the composition disclosed above or in any other form known to the person skilled in the art.

The composition or the composition for use according to the present invention comprises, in addition to the active ingredients specified above, at least one inert ingredient, such as at least one excipient among the ones commonly used and known to the person skilled in the art.

“Inert ingredient” means any substance, or combination of substances, auxiliary to the production of a pharmaceutical, dietary or nutraceutical form, which is to be found in the finished product and is not the active ingredient, although it can modify the stability, release or other features thereof.

Non-limiting examples of such ingredients, as is known to the person skilled in the art of formulations in the pharmaceutical, nutraceutical or food industry, are excipients such as diluents, absorbents, lubricants, colourants, surfactants, antioxidants, sweeteners, binders, disaggregating agents, anti-agglomerants, acidifying agents and the like.

In one preferred embodiment, the composition or the composition for use according to the present invention comprises or can comprise, in addition to the components disclosed above, at least one further active ingredient of natural or synthetic origin. Non-limiting examples of said active ingredients are vitamins, like vitamin B₁.

In one embodiment of the present invention, the composition as indicated above is for use in a method of treatment of a disorder or pathology associated with vascular calcification, which is at least one of aortic stenosis, hypertension, congestive heart failure, cardiac hypertrophy and cardiac ischaemia.

In one embodiment of the present invention, the composition as indicated above is for use in a subject with chronic kidney disease, diabetes, aortic stenosis and/or atherosclerosis, preferably in a subject with chronic kidney disease.

In the present invention, “chronic kidney disease”, or chronic kidney disease (CKD) is defined as a serious medical condition, characterized by progressive loss of kidney function over time. Chronic kidney disease is also defined as the presence of kidney damage, revealed by specific laboratory, instrumental or anatomical or pathological findings or reduced kidney function that has lasted at least 3 months, regardless of the basic pathology (national guide lines of Kidney Foundation, USA, 2017). The two main causes of CKD are diabetes with high blood pressure, which are responsible for two thirds of the cases.

An object of the present invention is, further, a method of treatment of vascular calcification and a disorder or pathology associated with vascular calcification comprising administering the composition of the invention to a subject in need thereof. Said disorder or pathology associated with vascular calcification is at least one of aortic stenosis, hypertension, congestive heart failure, cardiac hypertrophy and cardiac ischaemia.

The composition of the present invention, as defined above, can be a pharmaceutical composition, a foodstuff or a food supplement or a composition for a medical device.

An object of the present invention is a pharmaceutical composition, food supplement or composition for a medical device comprising or, alternatively, consisting of a composition (composition of the invention) comprising an effective amount of a mixture that comprises or, alternatively consists of at least (a) one K-group vitamin, (b) one inorganic magnesium salt and (c) one iron (III) oxide, complex or salt.

In the context of the present invention, the term “medical device” is used with the meaning according to Italian Legislative Decree no. 46 of 24 Feb. 1997, i.e. it indicates a substance or another product, whether used alone or in combination, intended by the manufacturer to be used for human beings for the purpose of diagnosis, prevention, monitoring, treatment or alleviation of a disease, and which does not exert its principal action in or on the human body for which it is intended, by pharmacological or immunological means or metabolic means, but which may be assisted in its function by such means.

The sucrosomial iron (Sucrosomiale®; Sucrosomial®), as defined above, can be solid or liquid, preferably it is in solid form.

The composition of the present invention as defined above, can be solid or liquid or semisolid, preferably it is in solid form.

The pharmaceutical composition, food supplement or composition for a medical device of the present invention can be solid, liquid or semi-solid, for example a suspension or gel, and can be in any form known to the person skilled in the sector of food, pharmaceutical or nutraceutical formulations, such as, by way of non-limiting example, in the form of a capsule, tablet, or powder that is at least partially dissolvable in the mouth or water soluble, granules, pellets or microparticles, optionally contained in a sachet or in a capsule (mini-tablet), liquid or semisolid preparation, gel, suspension, solution, two-phase liquid system and equivalent forms.

Preferably, the pharmaceutical composition, food supplement or composition for a medical device of the present invention is in solid form.

The pharmaceutical composition, food supplement or composition for a medical device according to the present invention preferably comprises the following components:

(a) vitamin K₂;

(b) magnesium oxide;

(c) iron pyrophosphate; and, optionally:

(d) sucresters E473;

(e) lecithin.

By way of non-limiting example, the composition according to the present invention can contain: Vitamin K₂: 30-180 micrograms (mcg).

Magnesium: from 50 mg to 450 mg; for example from 100 mg to 400 mg.

Iron: from 10 mg to 105 mg; for example from 10 mg to 30 mg.

An object of the present invention is further the non-therapeutic use of the composition of the present invention (as defined above) for non-therapeutic treatment of vascular calcification and of disorders and pathologies associated therewith in a subject requiring such non-therapeutic treatment.

Preferably, said disorders and pathologies associated with said vascular calcification are at least one of aortic stenosis, hypertension, congestive heart failure, cardiac hypertrophy and cardiac ischaemia.

Preferably, said non-therapeutic use of the composition of the present invention is indicated for a subject with chronic kidney disease, diabetes, aortic stenosis and/or atherosclerosis, preferably in a subject with chronic kidney disease.

Embodiments of the present invention are set out below (RPn)

RP1. A composition comprising or, alternatively, consisting of:
an effective amount of a mixture that comprises at least (a) one K-group vitamin or analogues and derivatives thereof, (b) one inorganic magnesium salt and (c) one iron (Ill) oxide, complex or salt
pharmaceutical or food grade excipients, additives and/or co-formulants for use in preventive or curative treatment of vascular calcification and the disorders and pathologies associated therewith in a subject, in which said use comprises administering the composition to said subject.

RP2. The composition for use according to RP1, in which at least one vitamin of the group is vitamin K₂.

RP3. The composition for use according to at least one of the preceding RPs, in which the inorganic magnesium salt is one of magnesium oxide, magnesium carbonate and magnesium chloride, or mixtures thereof, preferably magnesium oxide.

RP4. The composition for use according to at least one of the preceding RPs, in which the iron salt is iron pyrophosphate.

RP5. The composition for use according to RP4, in which the iron pyrophosphate is associated with sucrose fatty acid esters, i.e. sucresters (E473), lecithin and, optionally, pregelatinized starch.

RP6. The composition for use according to at least one of the preceding RPs, wherein said administration takes place via the oral route.

RP7. The composition for use according to at least one of the preceding RPs, in which the disorder or pathology associated with the vascular calcification is at least one of aortic stenosis, hypertension, congestive heart failure, cardiac hypertrophy and cardiac ischaemia.

RP8. The composition according to at least one of the preceding RPs, for use in a subject with chronic kidney disease, diabetes, aortic stenosis and/or atherosclerosis, preferably in a subject with chronic kidney disease.

RP9. A pharmaceutical composition, food supplement or medical device comprising
a composition comprising or, alternatively, consisting of an effective amount of a mixture that comprises at least (a) one K-group vitamin, (b) one inorganic magnesium salt and (c) one iron (111) oxide, complex or salt, and
pharmaceutical or food grade excipients, additives and/or co-formulants.

RP10. The pharmaceutical composition, food supplement or medical device according to RP9, comprising the following:
(a)—vitamin K₂;
(b)—magnesium oxide;
(c)—iron pyrophosphate; and, optionally:
(d) sucresters E473;
(e) lecithin.

The following experimental part provides examples of practical embodiments of the invention, without limiting the scope thereof.

### EXPERIMENTAL PART

(I) Various experiments were conducted in vitro to test the efficacy of the composition according to the present invention (comprising MgCl₂, Fe₄(P₂O₇)₃ and MK-7) on the slowing of the progression of the vascular calcification induced by high levels of sodium phosphate (Pi). The experimental model consists of vascular smooth muscle cells (VSMCs) removed from the aorta of a rat (primary cells) and stimulated for 7 days with Na₃PO₄ (Pi) 5 mM to induce them to calcify.

The calcification model entails adding Pi 5 mM and MgCl₂, Fe₄(P₂O₇)₃ treatments and MK-7 at the time-point at which the confluency of the culture cells exceeds 80%: this time-point is defined as day 0. On day 7, the effect of the treatment on the extracellular calcium deposition was analyzed both by a qualitative histological method (staining of calcium deposits by Alizarin Red Staining), and by a quantitative method, by measuring the amount of intracellular calcium by a spectrophotometric technique (FIG. 1). Each type of experiment was repeated at least 3 times and each condition was tested at least three times within every single experiment.

Initially, the effect of the individual compounds on the calcification, in particular MgCl₂ and Fe₄(P₂O₇)₃ was studied by analyzing the respective dose-response curves. Both treatments were powerful calcification inhibitors, with a dose-dependent protective effect (FIGS. 2, 3, 4, 5).

Afterwards, experiments were conducted to study the potential calcification-inhibiting effect of the co-administration of MgCl₂+Fe₄(P₂O₇)₃, combining both concentrations that are able to inhibit partially extracellular calcium deposition and concentrations that protected the cells almost totally from the progression of the calcification induced by Pi 5 mM. The results show that combining Fe₄(P₂O₇)₃ with MgCl₂ generates a synergic effect that retards VSMCs calcification compared with the single inhibiting effect of Fe₄(P₂O₇)₃ (FIGS. 6, 7, 8).

Subsequently, MK-7 was studied, adopting the same experimental approach previously used for MgCl₂ and Fe₄(P₂O₇)₃: although various experimental problems were encountered, including solubility in ethanol (recommended solvent), and the cellular toxicity of the vehicle, calcification inhibition was recorded ranging from 15 to 30% in ethyl acetate and of 20% in ethanol compared with compositions containing 5 mM of Pi in the same solvent (FIG. 9).

(II) Similarly to what has been stated for the in vitro (I) study, the efficacy was tested of the composition according to the present invention comprising sucrosomial iron pyrophosphate (SRM) (Sucrosomiale®; Sucrosomial®), vitamin K₂ menaquinone-7 (MK-7) and magnesium carbonate (MC) (composition called TRIO in Table 1 and FIG. 10) on the slowing of the progression of the vascular calcification induced by high levels of sodium phosphate (Pi) in a vascular cell culture and compared with the efficacy of the single components of the TRIO composition.

In particular, to execute the efficacy test, human aortic cell cultures were treated for 7 days with inorganic phosphate (Pi, 2 mM), a known calcifying agent, to induce mineralization of the cells, and with the products subjected to a concentration test 0.03% (% g of compound under examination/solution volume) and subsequently treated with the compound compositions under examination. At the end of the experimental treatment, the calcium content was quantified by a colorimetric assay, i.e. staining of the calcium deposits by Alizarin Red Staining (ARS).

The experimental design specified: untreated cell cultures (negative control CTR−), cell cultures (positive control CTR+) treated only with the calcifying agent (Pi) and cell cultures in which a calcification event was induced experimentally by the calcifying agent (Pi) and subsequently treated with the compositions/compounds under examination (0.03% concentrations) such as TRIO composition, only sucrosomial iron pyrophosphate (SRM) (Sucrosomiale®; Sucrosomial®), only vitamin K₂ menaquinone-7 (MK-7) and only magnesium carbonate (MC).

The Alizarin Red Staining S (ARS) is an anthraquinone staining solution that is widely used to evaluate calcium deposits in cell cultures. Alizarin Red Staining (ARS) is versatile because the dye can be easily extracted from the coloured single layer of the cells and can be promptly dosed. The ARS quantification assay provides a sensitive tool for partial quantification of calcium in a cell matrix. Mineralization is assessed by extracting the low pH calcified mineral, neutralization with ammonia hydroxide and 405 nm colorimetric detection.

In the present study, the cells subjected to the different aforementioned treatments were fixed for ARS and the deposition of mineral was quantified by a commercially available colorimetric kit. For this purpose, a calibration curve was plotted with known and increasing ARS concentrations.

The results are set out in Table 1 and FIG. 10 as an ARS quantity that is directly proportional to the calcium content in the different experimental treatments (average value±st. dev.) and as average % variation from the controls. The data obtained from the different experimental groups were analyzed statistically and compared with T-test. The significant values in FIG. 10 compared with the positive control (p<0.05) are marked with an asterisk (*).

TABLE 1

| | ARS mM | Standard | % VAR | % VAR |
|---|---|---|---|---|
| Treatment | average | deviation | vs CTR− | vs CTR+ |
| CTR− | 0.074 | 0.013 | | −68.9% |
| CTR+ (Pi 2 mM) | 0.238 | 0.005 | 221.1% | |
| 1 _TRIO composition | 0.091 | 0.008 | 23.1% | −61.6% |
| 3_SRM | 0.125 | 0.007 | 68.1% | −47.6% |
| 9_MK7 | 0.100 | 0.018 | 34.7% | 58.0% |
| 11_MC | 0.120 | 0.035 | 61.7% | −49.6% |

As the results of Table 1 and FIG. 10 show, the TRIO composition comprising sucrosomial iron pyrophosphate (SRM) (Sucrosomiale®; Sucrosomial®), vitamin K₂ menaquinone-7 (MK-7) and magnesium carbonate (MC) is more effective (−61.6%) in reducing the vascular calcification induced by sodium phosphate (Pi) in a vascular cell culture than are the single components of the mixture that were tested at the same mixture concentration (0.03%), proving to be a composition that significantly inhibits said induced mineralization process. Further, the data of Table 1 and FIG. 10 show the synergic effect of the single components of the composition against vascular calcification, the same concentration of the Trio composition being more effective than each of the single components.

1. A composition comprising:
an effective amount of a mixture that comprises or, alternatively, consisting of:
(a) a K-group vitamin, or analogues and derivatives thereof,
(b) an inorganic magnesium salt and
(c) an iron (Ill) oxide, complex or salt,
pharmaceutical or food grade excipients, additives and/or co-formulants for use in a preventive or curative method for treating vascular calcification in a subject, in which said use comprises administering the composition to said subject.

2. A composition for use according to claim 1, wherein said (c) iron (Ill) oxide, complex or salt is a (c) iron (Ill) oxide, complex or salt formulated in association with sucrose fatty acid esters or sucresters, lecithin and, optionally, gelatinized or pregelatinized starch (sucrosomial iron (Sucrosomiale®; Sucrosomial®)).

3. The composition for use according to any one of preceding claim, wherein said (a) K-group vitamin is vitamin K₂; preferably vitamin K₂ of the menaquinone 7 (MK7) subtype.

4. The composition for use according to any one of preceding claim, wherein said (b) inorganic magnesium salt is chosen from magnesium oxide, magnesium carbonate and magnesium chloride and mixtures thereof, preferably magnesium carbonate.

5. The composition for use according to any one of preceding claim, wherein said (c) iron (Ill) salt is iron pyrophosphate.

6. The composition for use according to any one of claims 1 to 4, wherein said (c) iron (Ill) salt is iron pyrophosphate formulated in association with sucrose fatty acid esters E473 or sucresters E473, lecithin E322, and, optionally, pregelatinized starch (sucrosomial iron (Sucrosomiale®; Sucrosomial®)).

7. A composition for use according to any one of claims 2 to 6, wherein said composition is for use in a preventive or curative method for treating disorders and pathologies associated with vascular calcification.

8. The composition for use according to claim 7, in which the disorder or pathology associated with vascular calcification is chosen from aortic stenosis, hypertension, congestive heart failure, cardiac hypertrophy and cardiac ischaemia.

9. The composition for use according to any one of preceding claim, wherein said composition is for use in a subject with chronic kidney disease, diabetes, aortic stenosis and/or atherosclerosis, preferably in a subject with chronic kidney disease.

10. The composition for use according to any one of preceding claim, wherein said administration takes place via the oral route.

11. A composition comprising:
an effective amount of a mixture comprising or, alternatively, consisting of:
(a) a K-group vitamin,
(b) an inorganic magnesium salt and
(c) an iron (Ill) oxide, complex or salt formulated in association with sucrose fatty acid esters or sucresters, lecithin and, optionally, gelatinized or pregelatinized starch (sucrosomial iron (Sucrosomiale®; Sucrosomial®)), and
pharmaceutical or food grade excipients, additives and/or co-formulants.

12. The composition according to claim 11, wherein said (a) K-group vitamin is vitamin K₂; preferably vitamin K₂ of the menaquinone 7 (MK7) subtype.

13. The composition according to claim 11 or 12, wherein said (b) inorganic magnesium salt is chosen from magnesium oxide, magnesium carbonate and magnesium chloride and mixtures thereof, preferably magnesium carbonate.

14. The composition according to any one of claims 11 to 13, wherein said (c) iron (Ill) salt is iron pyrophosphate formulated in association with sucrose fatty acid esters E473 or sucresters E473, lecithin E322, and, optionally, pregelatinized starch (sucrosomial iron (Sucrosomiale®; Sucrosomial®)).

15. The composition according to any one of claims 11 to 14, wherein said mixture comprises or, alternatively, consists of:
(a)—vitamin K₂;
(b)—magnesium carbonate;
(c)—iron pyrophosphate formulated in association with sucrose fatty acid esters or sucresters E473, lecithin E322, and, optionally, gelatinized or pregelatinized starch (sucrosomial iron (Sucrosomiale®; Sucrosomial®)).
